(19) United States
(12) Patent Application Publication
(10) Pub. No.: US 2022/0000660 A1
(43) Pub. Date: Jan. 6, 2022

(54) METHOD AND APPARATUS FOR SEPARATION OF TISSUE

(71) Applicant: EYEDEA MEDICAL, INC., Baltimore, MD (US)

(72) Inventors: Katherine SOLLEY, Baltimore, MD (US); Benjamin OSTRANDER, San Diego, CA (US); Christine DIAZ, Houston, TX (US); Ailon HAILEYESUS, Centreville, VA (US); Yueheng DOU, Fremont, CA (US); Edward RUPPEL, III, Saratoga, CA (US); Kunal PARIKH, Reynoldsburg, OH (US)

(21) Appl. No.: 17/294,264

(22) PCT Filed: Mar. 12, 2020
(86) PCT No.: PCT/US2020/022429

§ 371 Date: May 14, 2021

Related U.S. Application Data
(60) Provisional application No. 62/816,966, filed on Mar. 12, 2019.

(51) Int. Cl. A61F 9/007; A61B 90/11; A61B 90/14

(52) U.S. Cl. CPC A61F 9/007; A61B 2017/32035; A61B 90/14; A61B 90/11

(57) ABSTRACT

An ophthalmic medical device for separating layers of eye tissue is disclosed. The device may include a supporting structure for supporting the eye and the supporting structure may have at least one stabilizing feature that stabilizes the eye to reduce movement. The supporting structure may define at least one hollow channel for positioning a needle that is inserted into the cornea to make a fluid bubble that separates the Descemet's Membrane away from the underlying stroma. In some embodiments, the fluid bubble enables an easy, clean separation of the layers to later be transplanted in patients needing corneal transplants.

### TECHNICAL FIELD

The presently disclosed subject matter relates generally to the design of medical devices. More particularly, the present invention relates to the use of a fluid bubble to cleave tissue within the cornea or other tissue in the eye.

### BACKGROUND

The cornea consists of five main layers (anterior to posterior): the epithelium, Bowman's layer, the stroma, Descemet's Membrane, and the endothelium. The epithelium is the outermost layer of the surface of the eye, usually contains about five to seven layers of cells (50 microns thick), regenerates quickly, and acts as a barrier to the outside world. The Bowman's layer is a transition layer between the epithelium and stroma; this layer is usually only 8 to 14 microns thick and is largely involved in maintaining the proper shape of the cornea. The stroma is the bulk of the cornea, measuring around 500 microns thick and comprising of 90% of the cornea. This layer is filled with collagen fibers that are extremely regular in arrangement and constant in shape. The complex, interwoven structural organization of these fibers allows for the cornea to remain transparent. Descemet's Membrane is a thin layer that is secreted by the endothelium, which acts as a bed for the endothelial cells to rest on, only five to ten microns thick. The endothelium is a singular layer of cells, usually only five microns thick, that help maintain fluid equilibrium from inside the stroma. The main function of the endothelium is to pump out fluid so that the stroma remains optically clear and allow nutrients to enter the cornea from the aqueous humor. Finally, there is a controversial layer called Dua's Layer or the Pre-Descemet's Layer that is located directly anterior to the Descemet's Membrane. This layer is six to fifteen microns thick and is sometimes not considered to be a discrete new tissue “layer” in the sense that the other five have cellular differences and tissues planes.

The first corneal transplant occurred over 100 years ago in 1905 by Dr. Zirm, and consisting of a full thickness PK transplant in which all five layers of the cornea were replaced. Until the last twenty years, few advances had occurred in the corneal transplantation field. However, in the last two decades, doctors and researchers have found advantages to transplanting thinner layers of tissue, which has created a wider variety of corneal transplant types and techniques. More specifically, surgeons have started performing partial thickness endothelial keratoplasties (EKs), which consist of tissue grafts that are removed from the posterior cornea. The newest partial thickness transplants are Descemet's Stripping Automated Endothelial Keratoplasty (DSAEK), Descemet's Membrane Endothelial Keratoplasty (DMEK), Pre-Descemet's Endothelial Keratoplasty (PDEK), and Deep Anterior Lamellar Keratoplasty (DALK) or Anterior Lamellar Keratoplasty (ALK), which have all been implemented more widely in the last two decades. Each of these three EK techniques have advantages in comparison to others. With increasing variation of keratoplasty techniques, there has also been a shift from surgeon preparation to eye bank technician preparation of tissue grafts. This concept is known as pre-stripping tissue, and allows surgeons to save time in the operating room and reduce the likelihood of graft failure at the time of the surgery. This shift has been successful, but issues have arisen from eye bank technicians performing these delicate procedures.

The thickness of these endothelial keratoplasties vary significantly. DSAEK is the thickest partial thickness EK, usually 100-200 microns thick, with part of the posterior stroma, the Descemet's membrane and the endothelium transplanted. DMEK is the thinnest EK, usually around 10-15 microns, with only the Descemet's membrane and endothelium transplanted. PDEK is more similar to DMEK in that it is solely the pre-Descemet's layer, the Descemet's membrane, and the endothelium, usually ranging from 30-45 microns thick. In contrast to EK procedures, DALK or ALK procedures are used to replace the anterior side of the cornea and leave the patient's Descemet's Membrane and Endothelium intact.

Unlike DMEK and PDEK, DSAEK grafts are processed using an automated microkeratome device that enables eye bank technicians to prepare the tissue in a standardized and efficient manner. The technician uses a blade and pressurized chamber to determine the depth of the cut so that the graft thickness is within the correct range. In contrast, DMEK preparation within eye banks is done entirely by hand and is variable. The most common technique for preparing DMEK grafts is known as submerged cornea using backgrounds away (SCUBA). This technique requires the technician to carefully score a circle around the periphery of the cornea before peeling this membrane across the cornea. Alternatively, the technician can punch the cornea with a trephine to begin the separation of the DM from the stroma. This step is extremely delicate and difficult to master. On the other hand, PDEK is primarily made by inserting a needle into the cornea and injecting air to create the graft. Once this bubble is made, technicians punch the cornea with a trephine to obtain the correct diameter graft. This bubble technique has also been used by technicians to create a DMEK graft based on the same principle.

Although using the bubble or hydrodissection technique has shown superior outcomes in terms of length of training and preparation time, expanded donor pool, and other benefits, the technique in creation of a successful bubble has shown to be challenging in even experienced technicians. Likewise, the use of bubble in DALK procedures in the patient leads to better clinical outcomes due to a true cleavage between the stroma and the Descemet's Membrane. Two main issues arise during DMEK preparation: (a) the needle being inserted into the cornea does not enter in the correct plane, so too much fluid is injected into the stroma, and (b) pressure becomes too high within the cornea and endothelial cells begin to die. Similarly, in DALK procedures, there is significant risk of perforating the patient's Descemet's Membrane when the needle is inserted in the incorrect plane. Optimizing and standardizing these difficult aspects would allow wide adoption of this technique, both for DMEK preparation and in DALK surgery, and better DMEK preparation methods around the world.

Accordingly, there is a need for a standardized, superior method of corneal tissue separation. There is a desire for producing a high-quality tissue grafts in a consistent manner regardless of the technician's skill for DMEK. This needs to be done efficiently without tissue loss, with faster training, and with an increased number of potential donor corneas. This need is especially salient in places with lower resources and fewer corneal donations. Although highly skilled technicians can prepare tissue at high yields, less skilled technicians or eye banks with less resources have issues preparing tissue appropriately. Similarly, even highly skilled surgeons can perforate a patient's DM or not cleave the stromal/DM boundary properly in DALK surgery.

To reduce the risk of wasted tissue in DMEK preparation, more technicians and eye banks are examining the reasons for preparation failure and tissue loss. Eye banks understand that diabetic donors often make the tissue higher risk for preparation failure. This leads to exclusion of diabetic donor tissue in some eye banks for DMEK use.

Once grafts are processed by technicians, they need to pass Eye Bank Association of America (EBAA) standards in order to be sent for transplantation. The grafts will be checked for cell death, cell loss, and overall condition of the graft through evaluation from a specular microscope and slit-lamp biomicroscopy. Overall, the fewer the times the graft is moved or touched, the lower the likelihood for cell death or cell loss.

### BRIEF SUMMARY

The disclosed apparatus includes a stabilizing corneal base, pressure feedback system, and fluid injection system. The corneal base allows for optimal positioning of a needle that is inserted into the cornea to make a fluid bubble that separates the Descemet's Membrane away from the underlying stroma. The fluid bubble enables an easy, clean separation of the layers to facilitate better clinical outcomes in DALK and creation of grafts that will later be transplanted in patients needing corneal transplants.

The disclosed invention includes a number of embodiments of the system and methods for using the system. The supporting structure provides at least two useful functions: (a) to hold and stabilize the cornea or ocular tissue, and (b) to provide a needle tract or tunnel for the insertion of a needle or similar item. Likewise, a pressure feedback system provides at least two useful functions: (a) to measure pressure within the tubing tract and alert the technician if pressure become too high, and (b) to collect data on pressures within ocular tissue. The fluid injection system could comprise a syringe and/or a syringe pump. Syringe pumps that may be suitable in some applications are disclosed in the following United States patents all of which are hereby incorporated by reference herein: U.S. Pat. Nos. 9,113,843, 9,220,834, 9,241,641, 9,333,293, 9,352,105, and 9,457,140. This system controls the fluid entering through the needle into to ocular tissue. The disclosed invention optimizes the separation of layers within ocular tissue. The disclosed invention may also comprise additive features of the base, including a circular cutting attachment, a shipping attachment, and further tissue manipulation attachment.

The disclosed invention includes a number of different methods to use the system and separate the ocular tissues. Currently eye banks and surgeons are processing tissue manually with no assisted device. The disclosed invention includes the method of using a device to prepare the ocular tissue. There are no assistive, automated, or guided systems for preparing tissue. The invention describes methods for preparing ocular tissue for further use in surgery.

The disclosed invention will deskill and standardize tissue processing, improve accuracy and yields, and expand the donor pool while maintaining quality. The disclosed invention will also deskill separation of tissue within the eye in surgical settings, including DALK procedures. The device is an assistive, standardizing system that allows technicians and surgeons to more easily and quickly separate corneal tissue layers for use in surgery.

This device allows for reduce training time and processing time, increased yields, and expansion of the donor pool to high risk corneas. In low- and middle-income countries, technicians are often fearful of preparing certain corneal tissue grafts because of their lack of training and higher likelihood of failure. This fear prevents the technicians from even attempting preparation; therefore, the device would allow low-resource technicians to begin successfully processing tissue at high yields.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIGS. 1A, 1B, and 1C are views of disassembled components of one version of the apparatus of the present invention. FIG. 1A is a perspective, isometric view, showing many of the external features of the apparatus. FIG. 1B is a cross-sectional view of the three components of one embodiment of the apparatus, taken along section line 1B-1B seen in FIG. 1C. FIG. 1C is a side view, shown with the cross-sectional plane view of FIG. 1B.

FIGS. 2A, 2B, and 2C are views of assemble components of an apparatus of the present invention. FIG. 2A is a perspective, isometric view. FIG. 2B is a side view of the locked position of the three components of the apparatus, shown with the cross-sectional plane view of FIG. 2C. FIG. 2C is a cross-sectional view of the component, taken along 2C-2C of FIG. 2B.

FIGS. 3A, 3B, 3C, and 3D are views of assembled components of another version of the apparatus of the present invention. This embodiment of the apparatus highlights the tilted aspect of the present invention. FIG. 3A is a perspective, isometric view. FIG. 3B is a top view.

FIG. 3C is a side view of the components of the apparatus. FIG. 3D is the cross-sectional view of the components, taken along 3D-3D of FIG. 3C.

FIGS. 4A, 4B, 4C, and 4D are views of assembled components of another version of the apparatus of the present invention. These views illustrate a more simple, versatile embodiment of the apparatus. FIG. 4A is the perspective, isometric view. FIG. 4B is a top view. FIG. 4C is a side view of the components of the apparatus. FIG. 4D is the cross-sectional view of the components, taken along 4D-4D of FIG. 4C.

FIGS. 5A and 5B are photographs of embodiments of the systematic apparatus of the present invention. They illustrate the full system that can be combined to best prepare ocular tissue. FIG. 5A is a photograph of the main components of the more highly automated system on a benchtop setting. FIG. 5B is another embodiment of the components of the system on a benchtop setting.

FIG. 6A is a perspective view showing an ophthalmic medical device for separating layers of eye tissue in accordance with an example embodiment.

FIG. 6B is an exploded perspective view showing of the ophthalmic medical device shown in FIG. 6A.

FIG. 7A through FIG. 7C are elevation and plan views showing three sides of an ophthalmic medical device for separating layers of eye tissue in accordance with an example embodiment.

FIG. 8A is a top view showing an ophthalmic medical device for separating layers of eye tissue in accordance with an example embodiment.

FIG. 8B is a side view showing of the ophthalmic medical device shown in FIG. 8A.

FIG. 9 illustrates an ophthalmic medical device for separating layers of eye tissue.

FIG. 10A is a perspective view of an ophthalmic medical device 100 for separating layers of eye tissue. FIG. 10B is a cross-sectional view of the device shown in FIG. 10A. FIG. 10C is a perspective view of device shown in FIGS. 10A and 10B. FIG. 10D is a perspective view of device shown in FIGS. 10A and 10B.

FIG. 11 illustrates an ophthalmic medical device for separating layers of eye tissue.

FIG. 12 is an exploded perspective view showing an ophthalmic medical device for separating layers of eye tissue in accordance with an example embodiment.

FIG. 13A through FIG. 13C are elevation and plan views showing three sides of the base portion shown in FIG. 12.

FIG. 14A through FIG. 14F are elevation and plan views showing six sides of the stabilizing portion shown in FIG. 12.

While the embodiments of the disclosure are amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

The present disclosed invention has many possible embodiments. The specific embodiments shown herein should not be construed as the final embodiments of the apparatus, but simply examples of different variations of the apparatus. The present invention has a large scope in that this apparatus can come in many forms. Similarly, the methods are examples of potential ways that the apparatus can be handled and used to perform tissue separation. However, the method of using an apparatus to prepare certain ocular tissues is novel and has some variations.

The novel, ocular preparation system or apparatus of the disclosed invention includes the base 27, the needle 28, the sensor feedback system 25, and controlled fluid injection 24. The base 27 of the system is meant to fit ocular tissue, including a cornea, and needs to fit under a microscope. The base of the system comprises a base part 3, a stabilizing part 2, and can include a locking part 1. These parts assembled result in the base 27. A tunnel or tract 7 that can fit a needle is located in either the base part of the stabilizing part 2, and is one novel aspect of the present invention. Some embodiments have more features and details. All embodiments in the disclosed drawings have a base part 3 and a stabilizing part 2.

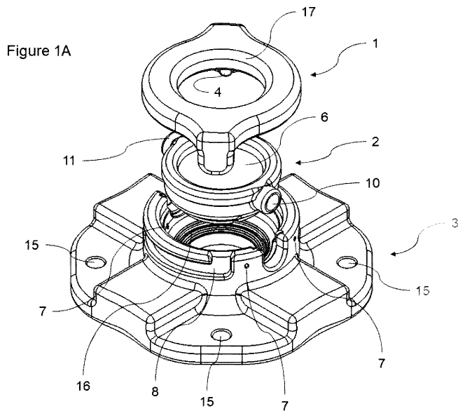

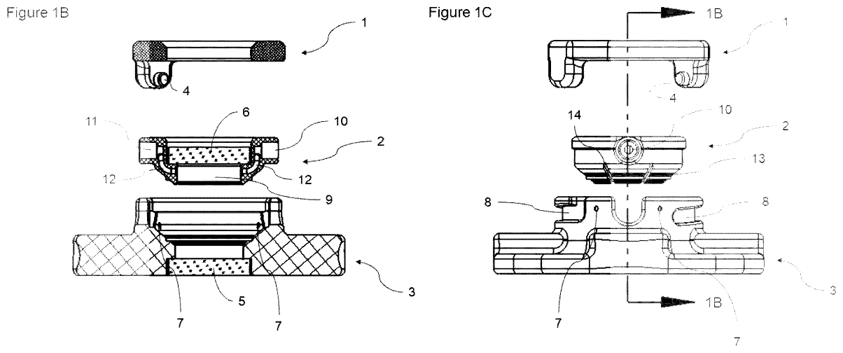

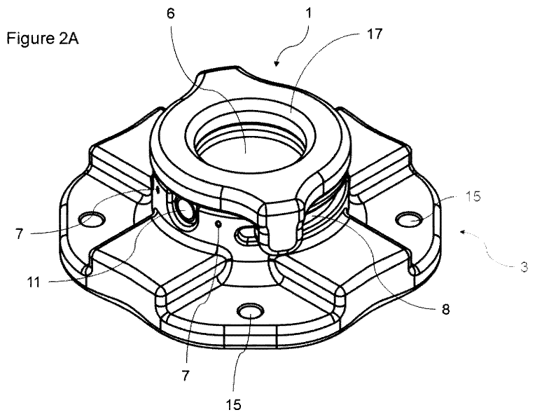

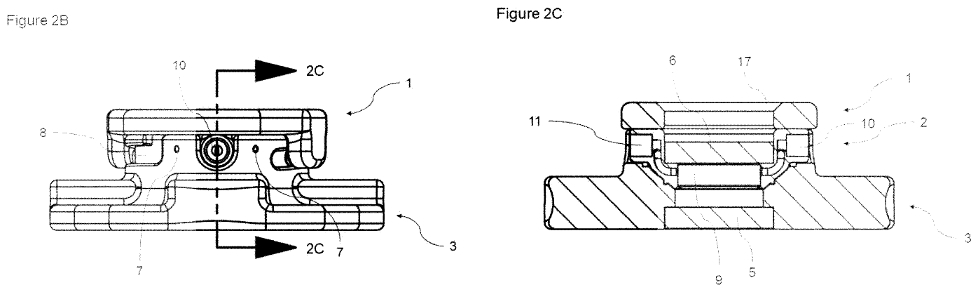

The disclosed FIGS. 1A-4D illustrate examples of embodiments of the base 27. One embodiment of the base 27 is illustrated in FIGS. 1A, 1B, 1C, 2A, 2B, and 2C. FIGS. 1A, 1B, and 1C illustrate the embodiment in the disassembled or unlocked position. FIGS. 2A, 2B, and 2C illustrate the embodiment in the assembled or locked position. In this embodiment, the cornea rests in the ridged concave surface 16 and is mechanical held in place by the stabilizing part 2. This both creates a mechanical force on the cornea, but also creates a small chamber 9 that fills with fluid and provides further pressure to the posterior cornea. The pegs 4 of the locking part 1 fits over the stabilizing part 2 and into the grooves 8 of the base part 3. The base part 3 is the foundation of the base 27 and rests on a flat surface, usually under a microscope. For stability, there are four holes 15 in the base part 3 that enable the technician to secure the part to a work surface. The two most essential aspects of this part 3 are (a) the ridged concave surface 16 for which the cornea rests and the tracts 7 that allow for optimal needle insertion into the cornea. Underneath the ridged concave surface 16 there is a hole that allows for slightly movement of the cornea in response to the pressure from the fluid-filled chamber 9. Underneath this hole, there is a circular clear material 5 that provides a window for the technician to observe the changes in the cornea from below. For stability of the cornea, there are grooved slots or tracts 8 that allow the peg 4 on the locking part 1 to slide along and increase force on the cornea.

The locking part 1 is the top piece that is designed with a peg 4 to fit over the stabilize part 2 and fit into the grooves 8 of the base part 3. This locking part enables a secure fit inside the fluid-filled chamber 9 by providing mechanical force on the stabilizing part 2. The fluid-filled chamber 9 provides pressure against the cornea that is lying posterior side up in the grooved 16, concave surface 21 that holds the cornea. This chamber 9 is filled with fluid (e.g. water, saline, BSS, Optisol, other tissue media) through the circular entrance opening 10 that can be attached to tubing. Air or other liquids already inside the chamber leave through the mirrored circular exit opening 11 through another tube. By changing the amount of fluid entering and leaving the chamber 9, the technician can set a specific pressure within chamber 9 to push and further secure the cornea. The stabilizing part 2 in FIGS. 1A, 1B, 1C, 2A, 2B, and 2C have two modes of stabilization: (a) stabilization from the mechanical force of placing the ridge concave structure 13 on the stabilizing part 2 over the cornea that is resting in the base part 3, and (b) stabilization from the fluidic forces from high pressures inside the chamber 9. When the stabilizing part 2 is pressure down by the locking part 1, the ridge surface 13 provides a force to the scleral rim around the donor cornea. The locking part 1 enable an extremely tight fit for the mechanical force, but also so that the chamber 9 does not leak.

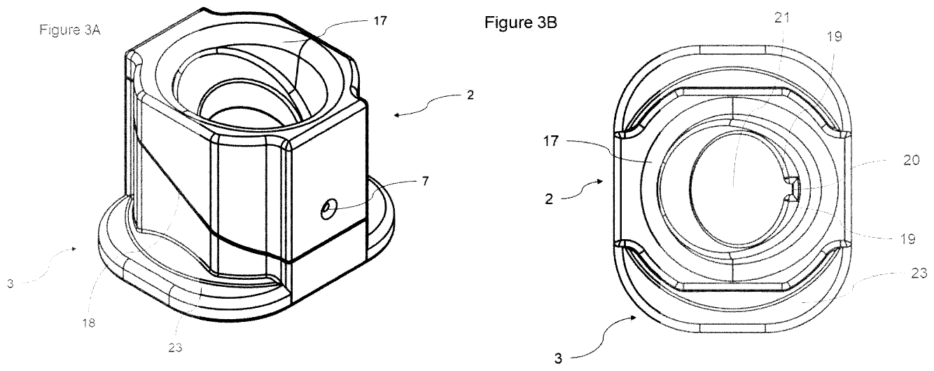

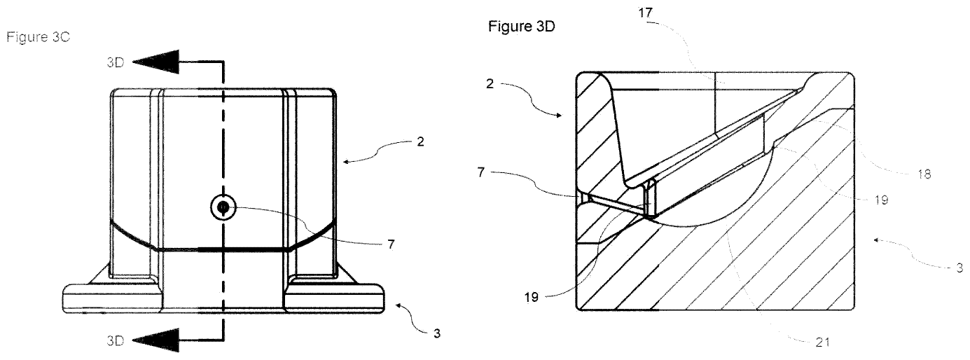

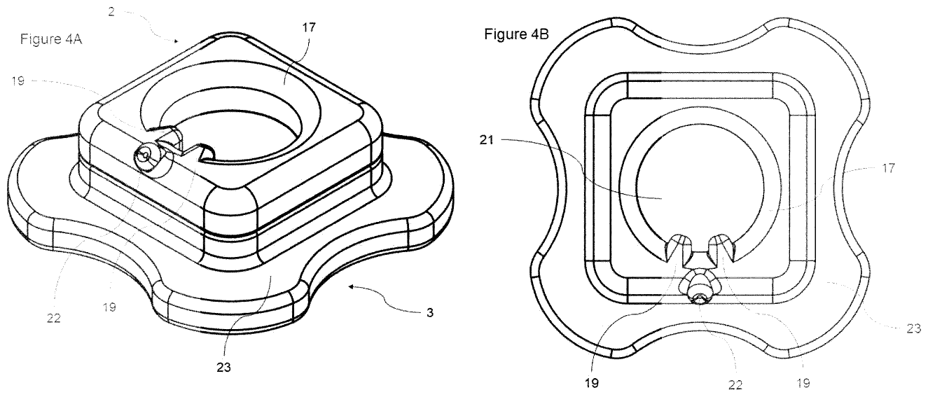

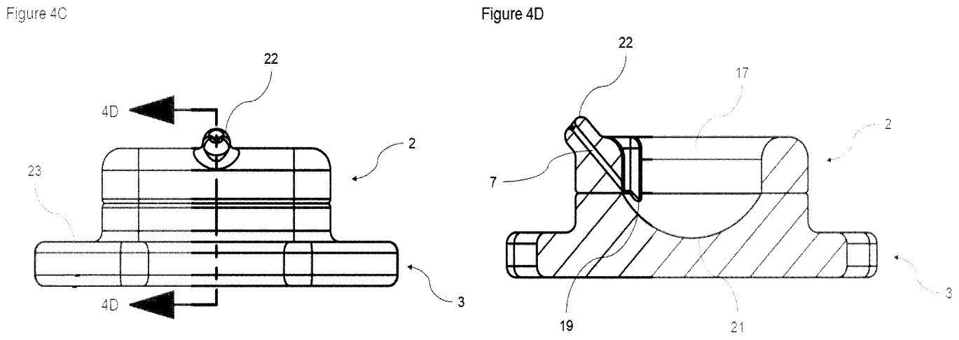

The stabilizing part 2 is the middle piece in FIGS. 1A-1C and 2A-2C and the top piece in FIGS. 3A-3D and 4A-4D. In FIGS. 1A-1C and 2A-2C, this piece has more functionality than just mechanical stabilization. The components for secondary pressure force is illustrated in FIGS. 1B and 2B. Fluid enters through one of the ports 10 and travels through the smaller channels 12 to fill the chamber 9. The cornea is positioned just beneath this chamber 9 while there is a clear circular material 6 that seals off the chamber 9. Unwanted air or other fluid exits out of the mirrored port 11 through the other small channel 12. Another key aspect of the stabilizing part 2 in the embodiment illustrated in FIGS. 1A-1C and 2A-2C is the grooved channels 14 that act as a continued needle tract.

These channels 14 in FIG. 1A-1C and 2A-2C or the needle tracts 7 in FIGS. 3A-3D and 4A-4D are lined up so that the needle 28 will enter the cornea at the limbus in a parallel or offset from parallel angle so the tip of the needle 28 is placed directly anterior to the Descemet's Membrane. The needle 28 is inserted at a controlled depth so that the bevel is at least 0.1 mm passed the limbus on the donor cornea. Once the needle 28 is in the correct plane, a controlled fluid injection 24 begins. This controlled fluid injection 24 can be in the form of pressurized fluid, a syringe, a syringe pump, or other embodiments of controlled fluid injection.

In another embodiment of the base 27, the connection between the stabilizing part 2 and the base part 3 is angled. This angled connection 18 allows the cornea to have some support on the opposing side from the needle tract 7. The angled connection 18 also allows for a more horizontal needle tract 7, which could allow for better usability and hand placement by the technicians during the procedure. Similarly, there is excess material 23 around the outside of most embodiments to aid in additional usability and ergonomics of the apparatus. In this embodiment of the base 27, there is a concave surface 21 where the cornea is placed for preparation prior to the placement of the stabilizing part 2. On the stabilizing part 2, there is a small ledge or holder 19 that presses down on the peripheral scleral rim to stabilize the cornea within the device. The small ledge 19 falls almost completely around the cornea, except a small space where the needle 25 comes out of the needle tract 7. This opening 20 serves to allow the technician to see the needle 25 entering the cornea.

In most embodiments of the present invention, there is an opening 17 above the cornea on the stabilizing part 2. This opening 17 allows for viewing of the cornea during preparation. However, in some embodiments, such as FIGS. 3A-3D and 4A-4D, these openings allow for a corneal trephine to cut the cornea after the bubble has been drained. This corneal trephine attachment to the base part 3 or through the stabilizing part 2 allows for ease of processing, cost savings, and potentially decreased cell loss due to corneal movement. This also allows for a better transition to pre-loaded tissue.

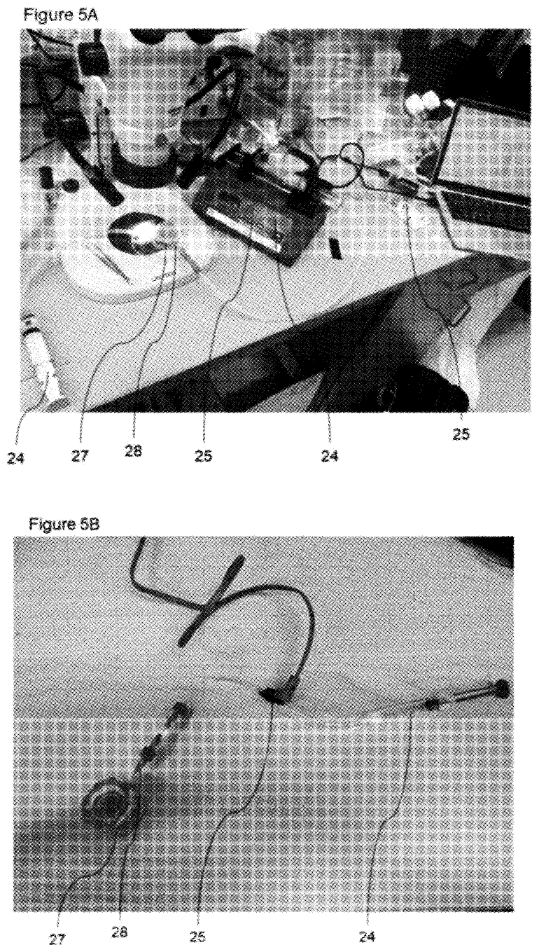

In some embodiments of the present invention, there is a sensor 25 that is included in this process. The sensor can be integrated in to different manners: (a) a force sensor 25 in FIG. 5A that is located between the driving force that is injecting the fluid or (b) an in-line pressure sensor 25 in FIG. 5B that is detected the pressure within the system. This sensor 25 will be able to detect large pressures within the cornea or large forces created within the controlled injection system 24 and notify the technician of the potential for tissue preparation failure. The technician can stop the preparation and restarted by rotating the cornea. Large pressures or forces indicate that the needle 25 is not in the correct position and is too deep into the stroma. The injection system 24 will inject 0.01 mL aliquots or more into the cornea up to at least 0.3 mL of fluid and until a bubble has formed to the desired size (at least 2 mm in diameter and at most the full diameter of the cornea). In the embodiment in FIGS. 1A-1C and 2A-2C, the pressure of the fluid in the chamber 9 will increase and the fluid within the bubble will immediately be drained. In other embodiments, the stabilizing part 2 will be removed and the technician will cut the periphery of the cornea to drain the fluid.

In another embodiment, illustrated in FIG. 4A-4D, the stabilizing part 2 will have a port 22 that allows for easier insertion of the needle 25. The concave surface 21 is back to its horizontal position and the angle of the needle tract 7 has shifted. The most ideal angle for needle tract has been found to be above 5 and below 50 degrees from a horizontal plane that the base 27 is resting on. Dowel pins and other fastening tools are used to stabilize the two parts of this other embodiment. The external features of the device around the excess material 23 are for aesthetics and usability for technicians.

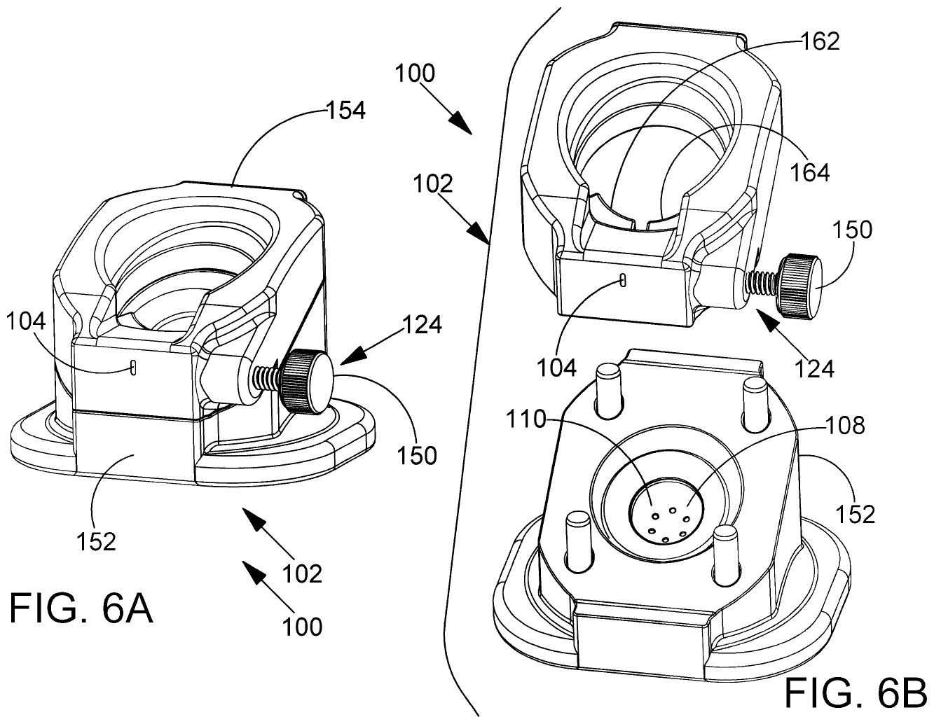

FIG. 6A is a perspective view showing an ophthalmic medical device 100 for separating layers of eye tissue in accordance with an example embodiment. FIG. 6B is an exploded perspective view showing of the ophthalmic medical device 100 shown in FIG. 6A. FIGS. 6A and 6B may be collectively referred to as FIG. 6. As illustrated in FIG. 6, the device 100 may comprise a supporting structure 102 for supporting the eye, with the supporting structure 102 having at least one feature that stabilizes the cornea to reduce movement. In the example embodiment of FIG. 6, the supporting structure 102 includes a base portion 152 and a stabilizing portion 154. A plurality of pins act to position the base portion 152 and the stabilizing portion 154 relative to one another in some embodiments. The base portion 152 of the supporting structure 102 has a concave surface 108 for receiving and/or holding a cornea in the example embodiment of FIG. 6. The concave surface 108 may define a concavity 110. In some embodiments, the supporting structure 102 of the device 100 includes at least one feature that stabilizes a cornea while the cornea is received in the concavity 110. In the example embodiment of FIG. 6, stabilizing portion 154 of the supporting structure 102 includes a first wing 162 and a second wing 164. In some embodiments, the first wing 162 and the second wing 164 apply stabilizing forces to a cornea while the cornea is received in the concavity 110. In some embodiments, the cornea is clamped or pinched between the wings and the concave surface 108 while the cornea is received in the concavity 110. In some embodiments, the cornea is trapped between the wings and the concave surface 108 while the cornea is received in the concavity 110. In some embodiments, the base portion 152 of the supporting structure 102 includes a first protrusion and a second protrusion that extending beyond the concave surface 108 into the concavity 110. In some embodiments, the first protrusion and the first wing 162 are configured and positioned so that the first protrusion and the first wing 162 are adjacent to one another when the supporting structure 102 is in an assembled state. In some embodiments, the first protrusion and the first wing 162 are configured and positioned so that a portion of a cornea is pinched and/or clamped between the first protrusion and the first wing 162 when the supporting structure 102 is in an assembled state with a cornea received in the concavity 110. In some embodiments, the second protrusion and the second wing 164 are configured and positioned so that the second protrusion and the second wing 164 are adjacent to one another when the supporting structure 102 is in an assembled state. In some embodiments, the second protrusion and the second wing 164 are configured and positioned so that a portion of a cornea is pinched and/or clamped between the second protrusion and the second wing 164 when the supporting structure 102 is in an assembled state with a cornea received in the concavity 110.

With reference to FIG. 6 it will be appreciated that the stabilizing portion 154 of the supporting structure 102 defines at a hollow channel 104. The hollow channel 104 is positioned between the first wing 162 and the second wing 164 in the example embodiment of FIG. 6. In some embodiments, the hollow channel 104 in positioned and dimensioned for guiding a needle. In the embodiment of FIG. 6, the device 100 includes a needle fixing mechanism 124 capable of selectively precluding movement of a needle relative to the hollow channel 104 during injection of a fluid. The needle fixing mechanism 124 includes a set screw 150 in the example embodiment of FIG. 6. In some embodiments, the needle fixing mechanism 124 includes at least one of a set screw, a locking pin, a quick-release pin, a spring pin, a pin or a screw.

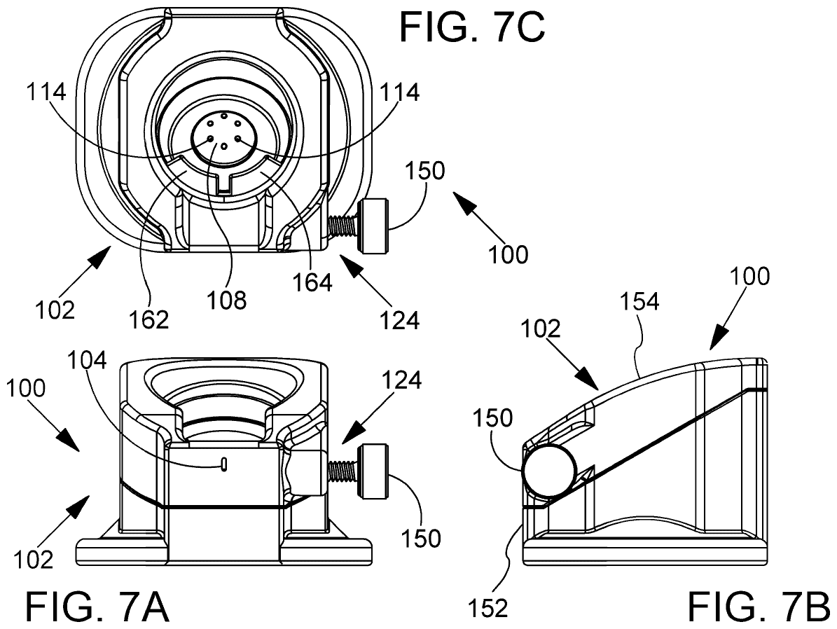

FIG. 7A through FIG. 7C are elevation and plan views showing three sides of an ophthalmic medical device 100 for separating layers of eye tissue in accordance with an example embodiment. Engineer graphics textbooks generally refer to the process used to create views showing six sides of a three-dimensional object as multiview projection or orthographic projection. It is customary to refer to multiview projections using terms such as front view, right side view, top view, rear view, left side view, and bottom view. In accordance with this convention, FIG. 7A may be referred to as a front view of the device 100, FIG. 7B may be referred to as a right side view of the device 100, and FIG. 7C may be referred to as a top view of the device 100. FIG. 7A through FIG. 7C may be referred to collectively as FIG. 7. Terms such as front view and top view are used herein as a convenient method for differentiating between the views shown in FIG. 7. It will be appreciated that the elements shown in FIG. 7 may assume various orientations without deviating from the spirit and scope of this detailed description. Accordingly, the terms front view, right side view, top view, and the like should not be interpreted to limit the scope of the invention recited in the attached claims.

The device 100 shown in FIG. 7 may comprise a supporting structure 102 for supporting the eye, with the supporting structure 102 having at least one feature that stabilizes the cornea to reduce movement. In the example embodiment of FIG. 7, the supporting structure 102 includes a base portion 152 and a stabilizing portion 154. The base portion 152 of the supporting structure 102 has a concave surface 108 defining a concavity 110 in the example embodiment of FIG. 7. In the example embodiment of FIG. 7, the features for stabilizing the cornea include the concave surface 108 for receiving the cornea and a plurality of holes 114 arranged in a pattern. In some embodiments, the holes 114 are selectively placed in communication with a source of sub-atmospheric pressure. In some embodiments, the holes 114 selectively apply sub-atmospheric pressure to a concave surface of the cornea. In some embodiments, the plurality of holes 114 are arranged to provide visual cues for centering the cornea relative to the concave surface 108. In some embodiments, the plurality of holes 114 are arranged in a pattern that defines a circle.

In the example embodiment of FIG. 7, the features for stabilizing the cornea also include a first wing 162 and a second wing 164 of the stabilizing portion 154. In some embodiments, the first wing 162 and the second wing 164 apply stabilizing forces to a cornea while the cornea is received in the concavity 110. In some embodiments, the cornea is clamped or pinched between the wings and the concave surface 108 while the cornea is received in the concavity 110. In some embodiments, the cornea is trapped between the wings and the concave surface 108 while the cornea is received in the concavity 110.

With reference to FIG. 7 it will be appreciated that the stabilizing portion 154 of the supporting structure 102 defines at a hollow channel 104. The hollow channel 104 is positioned between the first wing 162 and the second wing 164 in the example embodiment of FIG. 7. In some embodiments, the hollow channel 104 in positioned and dimensioned for guiding a needle. In the embodiment of FIG. 7, the device 100 includes a needle fixing mechanism 124 capable of selectively precluding movement of a needle relative to the hollow channel 104 during injection of a fluid. The needle fixing mechanism 124 includes a set screw 150 in the example embodiment of FIG. 7.

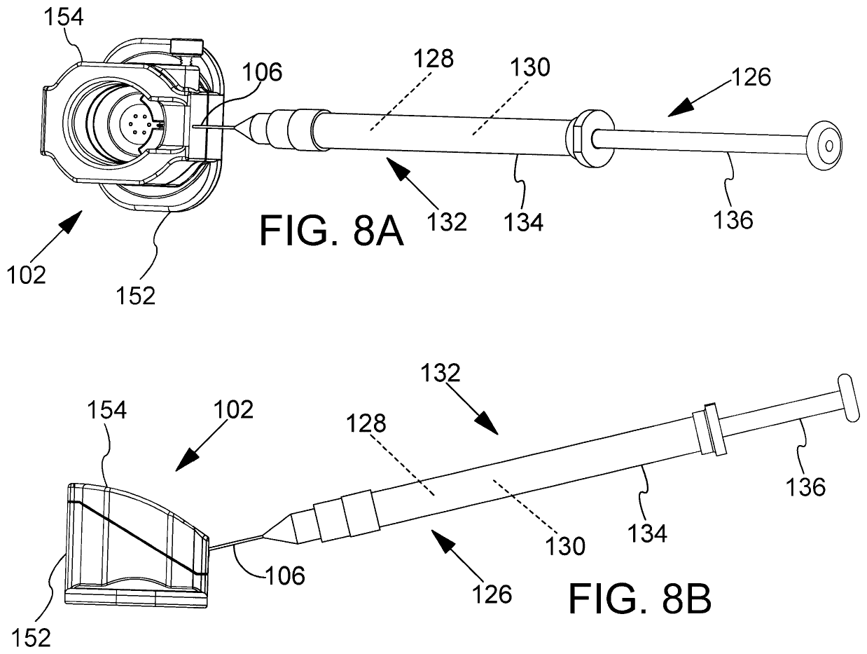

FIG. 8A is a top view showing an ophthalmic medical device 100 for separating layers of eye tissue in accordance with an example embodiment. FIG. 8B is a side view showing of the ophthalmic medical device 100 shown in FIG. 8A. FIGS. 8A and 8B may be collectively referred to as FIG. 8. In the example embodiment of FIG. 8, the device 100 comprises a supporting structure 102 including a base portion 152 and a stabilizing portion 154. In the example embodiment of FIG. 8, the stabilizing portion 154 of the supporting structure 102 defines at a hollow channel 104.

A needle 106 can be seen extending into the hollow channel 104 in FIG. 8. Some apparatus and methods in accordance with this detailed description may include and/or utilize a hypodermic needle with a sharp tip. Hypodermic needles that may be suitable in some applications are disclosed in the following United States patents all of which are hereby incorporated by reference herein: U.S. Pat. Nos. 2,560,162, 3,071,135, 3,308,822, 5,575,780, 6,009,933 and 6,517,523. In some embodiments, the hollow channel 104 is dimensioned and configured to receive a needle and guide the needle tip to a predetermined location inside the concavity 110 defined by the concave surface 108. In some embodiments, the hollow channel 104 is dimensioned and configured to receive a needle and guide the needle tip to a predetermined location inside tissue of an eye (or portion of the eye) while the cornea of the eye is received in the concavity 110 defined by the concave surface 108. In some embodiments, the hollow channel 104 is dimensioned and configured to receive of a 25, 26, 27, 28, 29, or 30 gauge needle and precisely guide the needle tip to a predetermined location. In some embodiments, the supporting structure 102 includes indicia indicating a needle size associated with that supporting structure 102.

Still referring to FIG. 8, in some embodiments, the hollow channel 104 in positioned and dimensioned for guiding the needle 106. In the example embodiment of FIG. 8, the device 100 also comprises a fluid injector 126 having a fluid cavity 128 and an injectable fluid 130 disposed in the fluid cavity 128. The fluid injector 126 comprises a syringe 132 in the example embodiment of FIG. 8. The syringe 132 may comprise a syringe barrel 134 and a syringe plunger 136, a distal portion of the syringe plunger 136 being slidingly received in the syringe barrel 134.

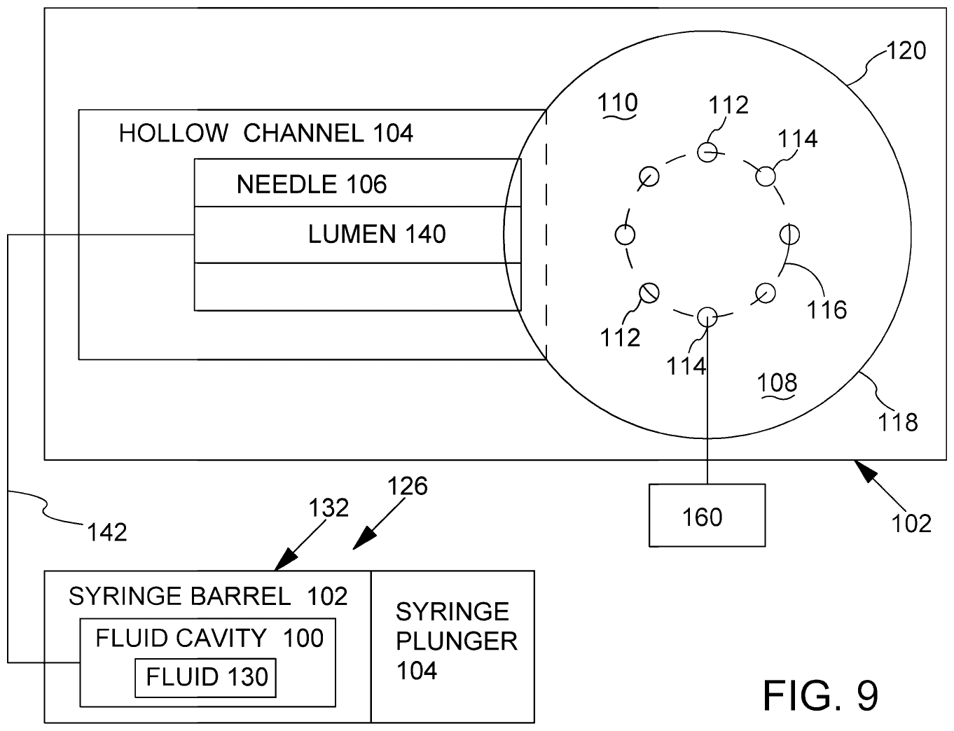

FIG. 9 illustrates an ophthalmic medical device 100 for separating layers of eye tissue. As illustrated in FIG. 9, the device 100 may comprise a supporting structure 102 for supporting a cornea or other eye tissue. In some embodiments, the supporting structure 102 has at least one feature that stabilizes the eye to reduce movement. In the example embodiment of FIG. 9, the supporting structure 102 defines at least one hollow channel 104. The at least one hollow channel 104 is positioned and dimensioned for guiding a needle 106 in the example embodiment of FIG. 9. In some embodiments, the hollow channel 104 has a length, a diameter and an aspect ratio of length to diameter, the aspect ratio of length to diameter being greater than 3. In some embodiments, the aspect ratio of length to diameter is greater than 6. In some embodiments, the aspect ratio of length to diameter is greater than 9. In some embodiments, the hollow channel 104 has a length greater than 2.5 mm. In some embodiments, the length of the hollow channel is greater than 5.0 mm. In some embodiments, the length of the hollow channel is greater than 7.5 mm. In some embodiments, the hollow channel has a diameter between 0.15 mm and 0.95 mm. In some embodiments, the hollow channel has a diameter between 0.25 mm and 0.55 mm.

The ophthalmic medical device 100 illustrated in FIG. 9 comprises a supporting structure 102 having a concave surface 108 for receiving and/or holding a cornea. The concave surface 108 defines a concavity 110 in the example embodiment of FIG. 9. The supporting structure 102 also includes one or more visual cues 112 for centering the cornea in the example embodiment of FIG. 9. In the example embodiment of FIG. 9, the one or more visual cues 112 for centering the cornea comprise a plurality of holes 114 arranged in a pattern, the plurality of holes 114 defining a first circle 116, the first circle 116 being concentric with a second circle 118 defined by an edge 120 of the concave surface 108. Still referring to FIG. 9, in some embodiments, an ophthalmic medical device 100 for separating layers of eye tissue comprises a supporting structure 102 having at least one feature that stabilizes the eye. In the example embodiment of FIG. 9, the stabilizing features of supporting structure 102 include a concave surface 108 for receiving a cornea and a plurality of holes 114 for applying a sub-atmospheric pressure to the convex side of the cornea. A source of sub-atmospheric pressure 160 may be selectively connected to the plurality of holes 114 in the example embodiment of FIG. 9.

The ophthalmic medical device 100 illustrated in FIG. 9 comprises a fluid injector 126 having a fluid cavity 128 and an injectable fluid 130 disposed in the fluid cavity 128. In some embodiments, the injectable fluid 130 is selected from the group consisting of water, air, corneal storage media, and saline. Examples of corneal storage medium that may be suitable in some applications include McCarey-Kaufman corneal storage medium, DEXSOL corneal storage media and OPTISOL corneal storage media which are all commercially available from Chiron Ophthalmics of Irvine, Calif. Examples of corneal storage medium that may be suitable in some applications also include LIFE4C (commercially available from Numedis, Inc. of Isanti, Minn.).

In some embodiments, an ophthalmic medical device 100 for separating layers of eye tissue includes a fluid injector 126 comprising a syringe 132. In some embodiments, the syringe 132 comprises a syringe barrel 134 and a syringe plunger 136, a distal portion of the syringe plunger 136 being slidingly received in the syringe barrel 134. In some embodiments, the syringe barrel 134 and the syringe plunger 136 cooperate to define the fluid cavity 128. In some embodiments, the device 100 also includes a needle 106 having a distal end, a proximal end and a needle body 138 extending between the distal end and the proximal end. In some embodiments, the needle body 138 defines a needle lumen 140 extending between the proximal end and the distal end of the needle 106. In some embodiments, the needle lumen 140 is in fluid communication with a fluid cavity 128. In some embodiments, the device 100 includes a conduit 142 operatively coupled to the syringe 132 and the needle 106, the conduit 142 placing the needle lumen 140 in fluid communication with the fluid cavity 128. In some embodiments, the device 100 includes a sensor 144. In some embodiments, the sensor 144 comprises at least one of a pressure sensor, a force sensor, a touch sensors, a flow sensor, and/or an accelerometer. In some embodiments, the sensor 144 comprises a pressure sensor placed in fluid communication with the needle lumen 140 and/or the fluid cavity 128 and the pressure sensor provides measurements suitable for use as feedback during fluid injection. In some embodiments, the device 100 includes an actuator that selectively causes the syringe 132 to inject fluid. Syringe actuators that may be suitable in some applications are disclosed in the following United States patents all of which are hereby incorporated by reference herein: U.S. Pat. Nos. 9,113,843, 9,220,834, 9,241,641, 9,333,293, 9,352,105, and 9,457,140.

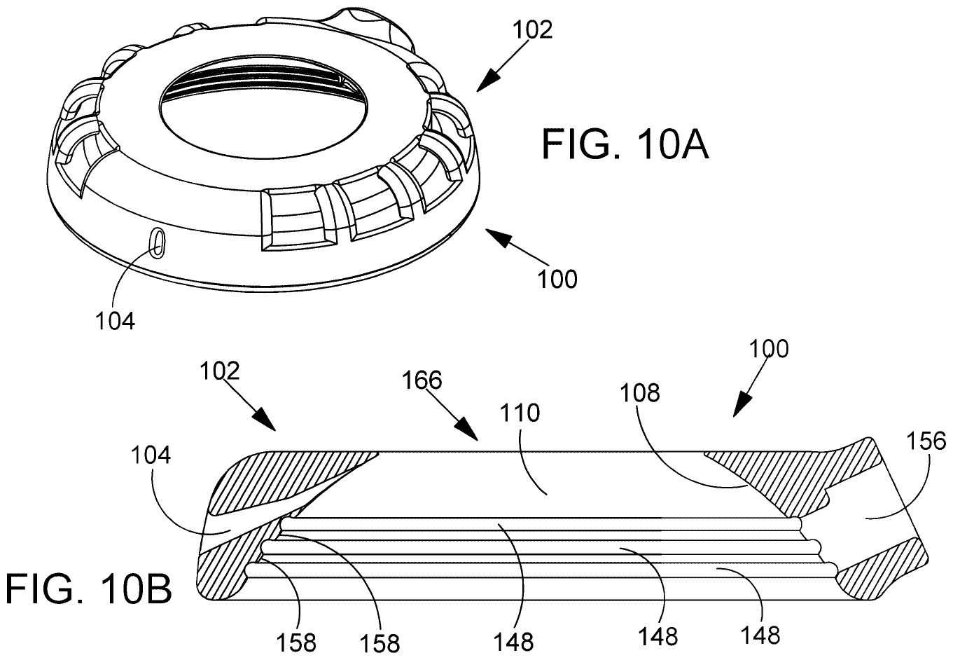

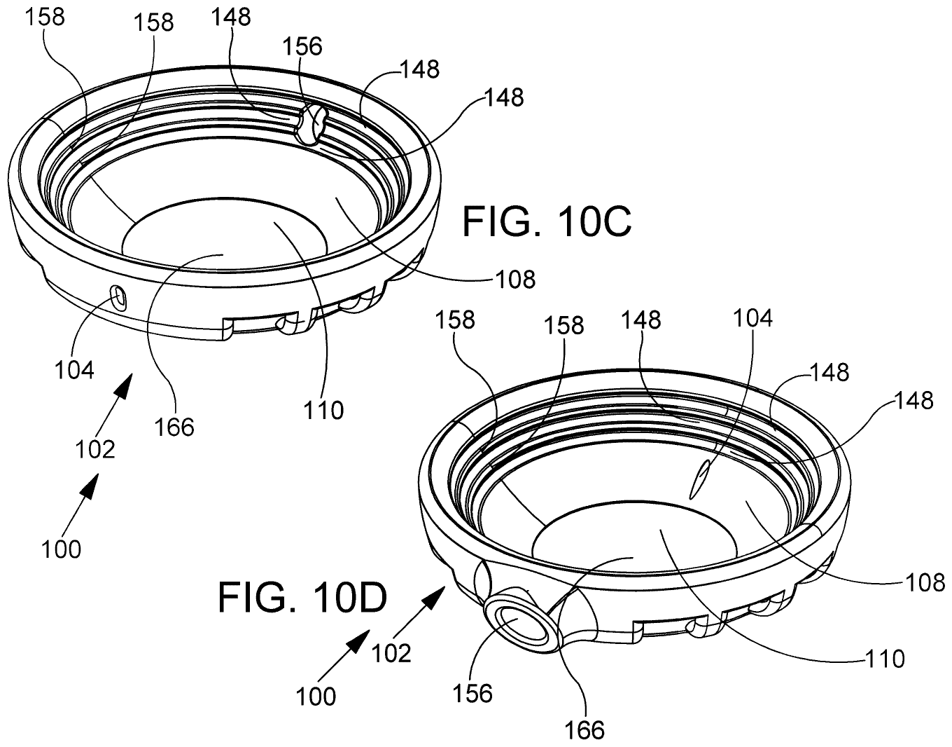

FIG. 10A is a perspective view of an ophthalmic medical device 100 for separating layers of eye tissue. FIG. 10B is a cross-sectional view of the device shown in FIG. 10A. FIG. 10C is a perspective view of device shown in FIGS. 10A and 10B. FIG. 10D is a perspective view of device shown in FIGS. 10A and 10B. FIGS. 10A through 10D may be collectively referred to as FIG. 10. The apparatus shown in FIG. 10 could be used, for example, in a DALK procedure. In the example embodiment of FIG. 10, the device 100 comprises a supporting structure 102 for supporting an eye. In the example embodiment of FIG. 10, the supporting structure includes a concave surface 108 that defines a concavity 110 for receiving a cornea of the eye. The ophthalmic medical device 100 illustrated in FIG. 10 comprises a supporting structure 102 including at least one stabilizing feature that stabilizes the eye to reduce movement. In the example embodiment of FIG. 10, the at least one stabilizing feature comprises a plurality of annular grooves 148 and annular ribs 158. Each of the annular ribs 158 is located between two annular grooves 148 in the example embodiment of FIG. 10. The concave surface 108 defines a concavity 110 and each of the annular grooves 148 opens into the concavity 110 in the example embodiment of FIG. 10. In the example embodiment of FIG. 10, each of the annular grooves 148 is in fluid communication with a port 156 that is defined by the supporting structure 102. A source of sub-atmospheric pressure 160 may be selectively connected to the port 156 and the plurality of annular grooves 148 in the example embodiment of FIG. 10. In the example embodiment of FIG. 10, the supporting structure 102 defines at least one hollow channel 104. The at least one hollow channel 104 is positioned and dimensioned for guiding a needle in the example embodiment of FIG. 10.

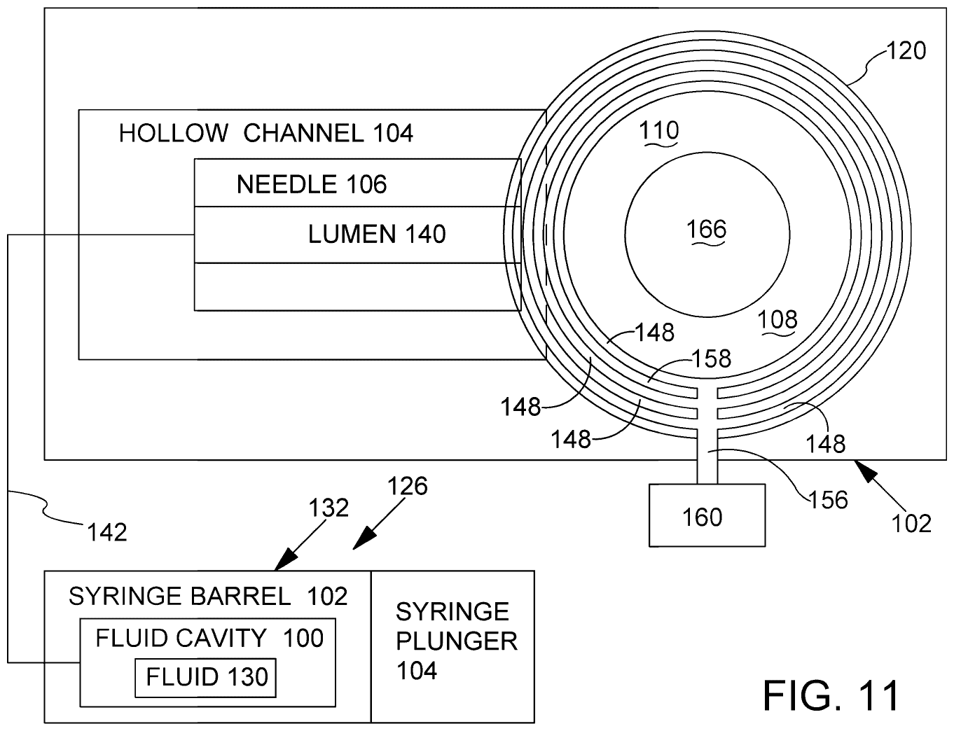

FIG. 11 illustrates an ophthalmic medical device 100 for separating layers of eye tissue. The apparatus illustrated in FIG. 11 could be used, for example, in a DALK procedure. As illustrated in FIG. 11, the device 100 may comprise a supporting structure 102 for supporting a cornea or other eye tissue. The ophthalmic medical device 100 illustrated in FIG. 11 comprises a supporting structure 102 having a concave surface 108 for receiving and/or holding a cornea and at least one stabilizing feature that stabilizes the eye to reduce movement. In the example embodiment of FIG. 11, the at least one stabilizing feature comprises a plurality of annular grooves 148 and annular ribs 158. Each of the annular ribs 158 is located between two annular grooves 148 in the example embodiment of FIG. 11. The concave surface 108 defines a concavity 110 and each of the annular grooves 148 opens into the concavity 110 in the example embodiment of FIG. 11. In the example embodiment of FIG. 11, each of the annular grooves 148 is in fluid communication with a port 156 that is defined by the supporting structure 102. A source of sub-atmospheric pressure 160 may be selectively connected to the port 156 and the plurality of annular grooves 148 in the example embodiment of FIG. 11. An edge 120 of the concave surface 108 is visible in FIG. 11.

The ophthalmic medical device 100 illustrated in FIG. 11 also comprises a fluid injector 126 having a fluid cavity 128 and an injectable fluid 130 disposed in the fluid cavity 128. In the example embodiment of FIG. 11, the fluid injector 126 comprising a syringe 132 having a syringe barrel 134 and a syringe plunger 136. A distal portion of the syringe plunger 136 may be slidingly received in the syringe barrel 134. The syringe barrel 134 and the syringe plunger 136 may cooperate to define the fluid cavity 128. With reference to FIG. 11, it will be appreciated that device 100 also includes a needle 106 defining a needle lumen 140. The needle lumen 140 is in fluid communication with the fluid cavity 128 in the embodiment of FIG. 11. In FIG. 11, a conduit 142 can be seen operatively coupled between the syringe 132 and the needle 106 so that the conduit 142 can place the needle lumen 140 in fluid communication with the fluid cavity 128. With reference to FIG. 11, it will be appreciated that the needle 150 is disposed inside a hollow channel 104 defined by the supporting structure 102. In some embodiments, at least one hollow channel 104 is positioned and dimensioned for guiding the needle 106.

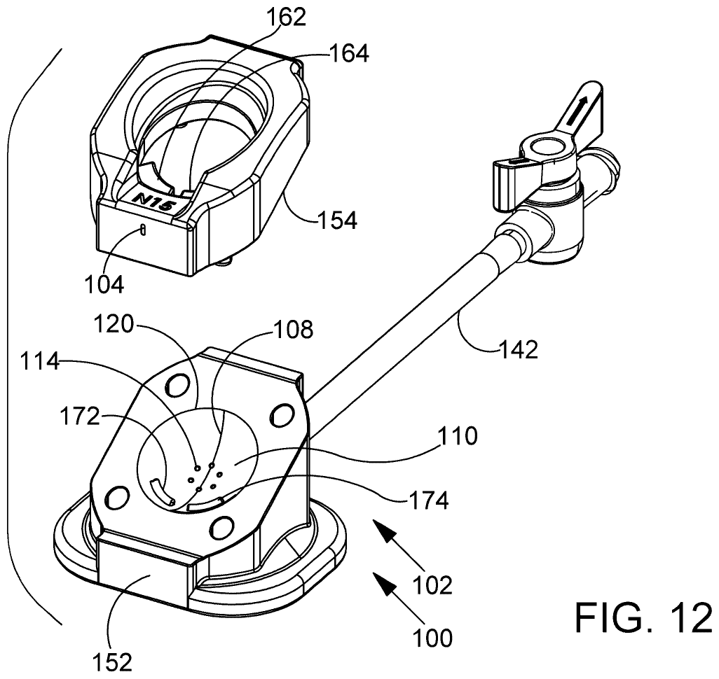

FIG. 12 is an exploded perspective view showing an ophthalmic medical device 100 for separating layers of eye tissue in accordance with an example embodiment. As illustrated in FIG. 12, the device 100 may comprise a supporting structure 102 for supporting the eye, with the supporting structure 102 having at least one feature that stabilizes the cornea to reduce movement. In the example embodiment of FIG. 12, the supporting structure 102 includes a base portion 152 and a stabilizing portion 154. A plurality of pins act to position the base portion 152 and the stabilizing portion 154 relative to one another in some embodiments. The base portion 152 of the supporting structure 102 has a concave surface 108 defining a concavity 110. The concavity may be dimensioned and configured for receiving and/or holding a cornea. In FIG. 12, a first protrusion 172 and a second protrusion can be seen extending beyond the concave surface 108.

Still referring to FIG. 12, in some embodiments, the supporting structure 102 of the device 100 includes at least one feature that stabilizes a cornea while the cornea is received in the concavity 110. In the example embodiment of FIG. 12, the base portion 152 of the supporting structure 102 includes the first protrusion 172 and the second protrusion 174 that can be seen extending beyond the concave surface 108. In the example embodiment of FIG. 12, the stabilizing portion 154 of the supporting structure 102 also includes a first wing 162 and a second wing 164. In some embodiments, the first protrusion 172 and the first wing 162 are configured and positioned so that the first protrusion 172 and the first wing 162 are adjacent to one another when the supporting structure 102 is in an assembled state. In some embodiments, the first protrusion 172 and the first wing 162 are configured and positioned so that a portion of a cornea is pinched and/or clamped between the first protrusion 172 and the first wing 162 when the supporting structure 102 is in an assembled state with a cornea received in the concavity 110. In some embodiments, the second protrusion 174 and the second wing 164 are configured and positioned so that the second protrusion 174 and the second wing 164 are adjacent to one another when the supporting structure 102 is in an assembled state. In some embodiments, the second protrusion 174 and the second wing 164 are configured and positioned so that a portion of a cornea is pinched and/or clamped between the second protrusion 174 and the second wing 164 when the supporting structure 102 is in an assembled state with a cornea received in the concavity 110.

With reference to FIG. 12 it will be appreciated that the stabilizing portion 154 of the supporting structure 102 defines at a hollow channel 104. The hollow channel 104 is positioned between the first wing 162 and the second wing 164 in the example embodiment of FIG. 12. In some embodiments, the hollow channel 104 in positioned and dimensioned for guiding a needle. In the example embodiment of FIG. 12, the features for stabilizing the cornea include the concave surface 108 for receiving the cornea and a plurality of holes 114 arranged in a pattern. In some embodiments, the holes 114 are selectively placed in communication with a source of sub-atmospheric pressure via a conduit 142. In some embodiments, the holes 114 selectively apply sub-atmospheric pressure to a concave surface of the cornea. In some embodiments, the plurality of holes 114 are arranged to provide visual cues for centering the cornea relative to the concave surface 108. In some embodiments, the plurality of holes 114 are arranged in a pattern that defines a circle.

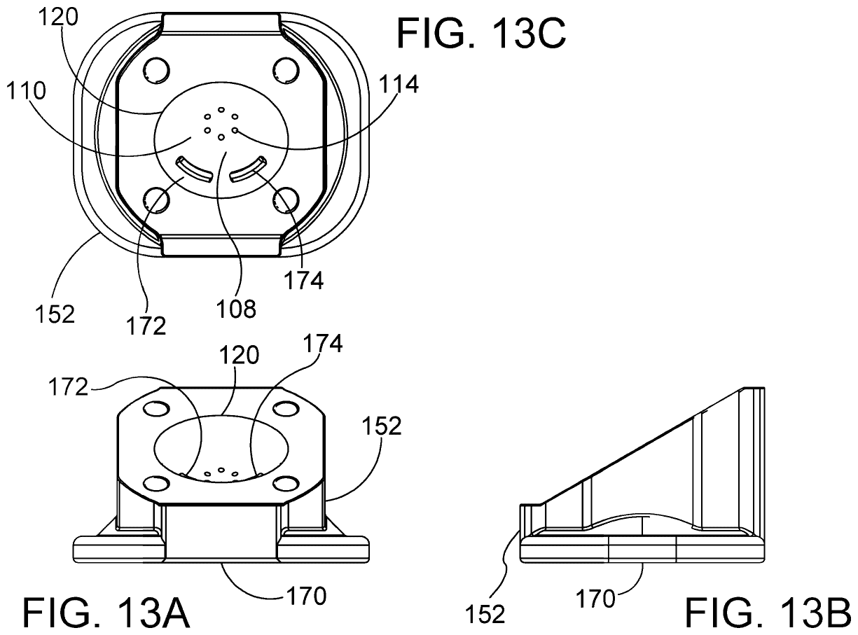

FIG. 13A through FIG. 13C are elevation and plan views showing three sides of the base portion 152 shown in FIG. 12. Engineer graphics textbooks generally refer to the process used to create views showing six sides of a three-dimensional object as multiview projection or orthographic projection. It is customary to refer to multiview projections using terms such as front view, right side view, top view, rear view, left side view, and bottom view. In accordance with this convention, FIG. 13A may be referred to as a front view of the base portion 152, FIG. 13B may be referred to as a right side view of the base portion 152, and FIG. 13C may be referred to as a top view of the base portion 152. FIG. 13A through FIG. 13C may be referred to collectively as FIG. 13. Terms such as front view and top view are used herein as a convenient method for differentiating between the views shown in FIG. 13. It will be appreciated that the elements shown in FIG. 13 may assume various orientations without deviating from the spirit and scope of this detailed description. Accordingly, the terms front view, right side view, top view, and the like should not be interpreted to limit the scope of the invention recited in the attached claims.

With reference to FIG. 13 it will be appreciated that the base portion 152 includes a first protrusion 172 and a second protrusion 174 that can be seen extending beyond a concave surface 108. The concave surface 108 defines a concavity 110 in the embodiment of FIG. 13. A plurality of holes 114 extending through the concave surface 108 and fluidly communicate with the concavity 110 in the embodiment of FIG. 13. The concave surface may extend to an edge 120. In the example embodiment of FIG. 13, the edge 120 of the concave surface 108 defines a reference plane that extends at an angle less than 90 degrees in relation to a base plane defined by a bottom surface 170 the base portion 152.

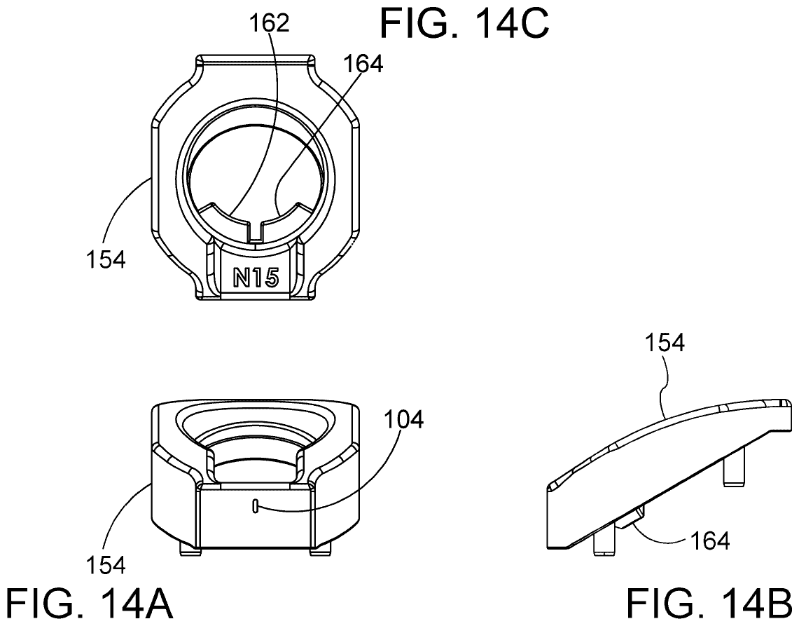

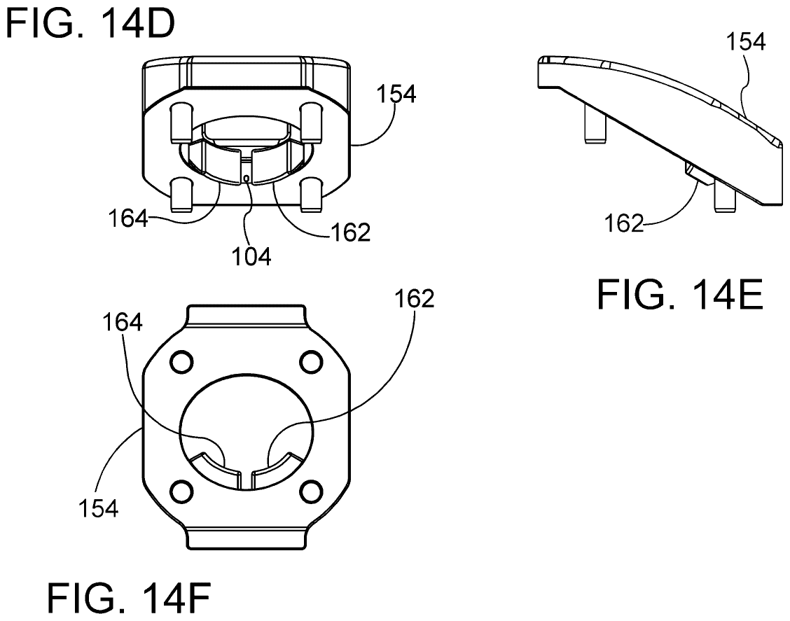

FIG. 14A through FIG. 14F are elevation and plan views showing six sides of the stabilizing portion 154 shown in FIG. 12. Engineer graphics textbooks generally refer to the process used to create views showing six sides of a three-dimensional object as multiview projection or orthographic projection. It is customary to refer to multiview projections using terms such as front view, right side view, top view, rear view, left side view, and bottom view. In accordance with this convention, FIG. 14A may be referred to as a front view of the stabilizing portion 154, FIG. 14B may be referred to as a right side view of the stabilizing portion 154, and FIG. 14C may be referred to as a top view of the stabilizing portion 154. FIG. 14A through FIG. 14C may be referred to collectively as FIG. 14. Terms such as front view and top view are used herein as a convenient method for differentiating between the views shown in FIG. 14. It will be appreciated that the elements shown in FIG. 14 may assume various orientations without deviating from the spirit and scope of this detailed description. Accordingly, the terms front view, right side view, top view, and the like should not be interpreted to limit the scope of the invention recited in the attached claims. FIG. 14D may be referred to as a rear view of the stabilizing portion 154, FIG. 14E may be referred to as a left side view of the stabilizing portion 154, and FIG. 14F may be referred to as a bottom view of the stabilizing portion 154.

With reference to FIG. 14 it will be appreciated that the stabilizing portion 154 includes a first wing 162 and a second wing 164. With reference to FIG. 14 it will also be appreciated that the stabilizing portion 154 of the supporting structure 102 defines at a hollow channel 104. The hollow channel 104 can be seen positioned between the first wing 162 and the second wing 164 in FIG. 14D. In some embodiments, the hollow channel 104 is dimensioned and configured to receive of a 25, 26, 27, 28, 29, or 30 gauge needle and precisely guide the needle tip to a predetermined location. In some embodiments, the supporting structure 102 includes indicia indicating a needle size associated with that supporting structure 102.

Referring to FIG. 6 through FIG. 14, in some embodiments, an ophthalmic medical device 100 for separating layers of eye tissue comprises a supporting structure 102 having a concave surface 108 defining a concavity 110. In some embodiments, the supporting structure 102 defines a hollow channel 104 that is disposed in fluid communication with the concavity 110. In some embodiments, the concave surface 108 and the concavity 110 are dimensioned and configured for receiving and/or holding a cornea. In some embodiments, the hollow channel 104 is dimensioned and configured to receive a needle and guide the needle tip to a predetermined location inside the concavity 110 defined by the concave surface 108. In some embodiments, the hollow channel 104 is dimensioned and configured to receive of a 25, 26, 27, 28, 29, or 30 gauge needle and precisely guide the needle tip to a predetermined location.

In some embodiments, the hollow channel 104 is dimensioned and configured to receive a needle and guide the needle tip to a predetermined location inside tissue of an eye (or portion of the eye) while the cornea of the eye is received in the concavity 110 defined by the concave surface 108. In some embodiments, the hollow channel 104 is dimensioned and configured to receive a needle and guide the needle tip to a predetermined location proximate a distal edge of the peripheral cornea of an eye. In some embodiments, the hollow channel 104 is dimensioned and configured to receive a needle and guide the needle tip to a predetermined location inside the peripheral cornea of an eye. In some embodiments, the hollow channel 104 is dimensioned and configured to receive a needle and guide the needle tip to a location near where scleral tissue meets a distal edge of the peripheral cornea of an eye. In some embodiments, the hollow channel 104 is dimensioned and configured to receive a needle and guide the needle tip to a location between the iris and the trabecular meshwork. In some embodiments, the hollow channel 104 has a length, a diameter and an aspect ratio of length to diameter, the aspect ratio of length to diameter being greater than 3. In some embodiments, the aspect ratio of length to diameter is greater than 6. In some embodiments, the aspect ratio of length to diameter is greater than 9. In some embodiments, the hollow channel 104 has a length greater than 2.5 mm. In some embodiments, the length of the hollow channel is greater than 5.0 mm. In some embodiments, the length of the hollow channel is greater than 7.5 mm. In some embodiments, the hollow channel has a diameter between 0.15 mm and 0.95 mm. In some embodiments, the hollow channel has a diameter between 0.25 mm and 0.55 mm.

Still referring to FIG. 6 through FIG. 14, in some embodiments, an ophthalmic medical device 100 for separating layers of eye tissue comprises a supporting structure 102 having a concave surface 108 for receiving and/or holding a cornea. The concave surface 108 may define a concavity 110. In some embodiments, the supporting structure 102 includes one or more visual cues 112 for centering the cornea. In some embodiments, the one or more visual cues 112 for centering the cornea comprise a plurality of holes 114 arranged in a pattern, the plurality of holes defining a first circle 116, the first circle 116 being concentric with a second circle 118 defined by an edge 120 of the concave surface 108. In some embodiments, the supporting structure 102 has a concave surface 108 that has at least a 1% tilt from a horizontal reference plane.

Still referring to FIG. 6 through FIG. 14, in some embodiments, an ophthalmic medical device 100 for separating layers of eye tissue comprises a supporting structure 102 having at least one feature that stabilizes the eye. In some embodiments, the stabilizing feature includes the locking mechanism that applies a mechanical force across a cornea contacting region, the cornea contacting region extending through a span that is equal to or less than 360 degrees around the periphery of a cornea. In some embodiments, the stabilizing features of the supporting structure 102 apply a sub-atmospheric pressure on a convex side of a cornea. In some embodiments, the stabilizing features of the supporting structure 102 comprises a plurality of holes 114 and a sub-atmospheric pressure is applied to the convex side of a cornea via the plurality of holes 114. In some embodiments, the stabilizing features apply a pressure on a concave side of a cornea. In some embodiments, the stabilizing features apply a positive pressure (e.g., a pressure greater than atmospheric pressure) on a concave side of a cornea. In some embodiments, the stabilizing features of the support structure 102 comprise a concave surface 108 for receiving a cornea and a plurality of holes 114 arranged in a pattern, the plurality of holes 114 defining a first circle 116. In some embodiments, the first circle 116 is concentric with a second circle 118 defined by an edge 120 of the concave surface 108. In some embodiments, the holes 114 are in selective communication with a source of sub-atmospheric pressure. In some embodiments, the stabilizing features of the support structure 102 comprise a concave surface 108 for receiving a cornea and at least one annular groove 148 disposed about the concave surface 108. In some embodiments, the annular groove 148 extends 360 degrees along a circular path around the concave surface 108. In some embodiments, the annular groove 148 is in selective communication with a source of sub-atmospheric pressure.

Still referring to FIG. 6 through FIG. 14, in some embodiments, the hollow channel 104 extends along a line that is parallel to a tangent line, the tangent line being tangent to a concave surface 108, the concave surface 108 being dimensioned and configured for receiving and/or holding a cornea. In some embodiments, the hollow channel 104 is placed at an angle less than or equal to 90 degrees in relation to a reference plane, the reference plane being defined by a circular opening of a concavity 110, the concavity 110 being dimensioned and configured for receiving and/or holding a cornea. In some embodiments, the hollow channel comprises a curved needle tract. In some embodiments, the hollow channel comprises a straight needle tract.

Referring to FIG. 6 through FIG. 8, in some embodiments, an ophthalmic medical device 100 for separating layers of eye tissue comprises a needle fixing mechanism 124 capable of selectively precluding movement of a needle relative to the hollow channel 104 during injection of a fluid. In some embodiments, the needle fixing mechanism 124 includes at least one of a set screw, a locking pin, a quick-release pin, a spring pin, a pin or a screw.

Referring to FIG. 9 and FIG. 11, in some embodiments, an ophthalmic medical device 100 for separating layers of eye tissue comprises a fluid injector 126 having a fluid cavity 128 and an injectable fluid 130 disposed in the fluid cavity 128. In some embodiments, the injectable fluid 130 is selected from the group consisting of water, saline, air, corneal storage media, and balanced salt solution (BSS). Examples of corneal storage medium that may be suitable in some applications include McCarey-Kaufman corneal storage medium, DEXSOL corneal storage media and OPTISOL corneal storage media which are all commercially available from Chiron Ophthalmics of Irvine, Calif. Examples of corneal storage medium that may be suitable in some applications also include LIFE4C (commercially available from Numedis, Inc. of Isanti, Minn.).

Referring to FIG. 8, FIG. 9 and FIG. 11, in some embodiments, an ophthalmic medical device 100 for separating layers of eye tissue includes a fluid injector 126 comprising a syringe 132. In some embodiments, the syringe 132 comprises a syringe barrel 134 and a syringe plunger 136, a distal portion of the syringe plunger 136 being slidingly received in the syringe barrel 134. In some embodiments, the syringe barrel 134 and the syringe plunger 136 cooperate to define the fluid cavity 128. In some embodiments, the device 100 also includes a needle 106 having a distal end, a proximal end and a needle body 138 extending between the distal end and the proximal end. In some embodiments, the needle body 138 defines a needle lumen 140 extending between the proximal end and the distal end of the needle 106. In some embodiments, the needle lumen 140 is in fluid communication with a fluid cavity 128. In some embodiments, the device 100 includes a conduit 142 operatively coupled to the syringe 132 and the needle 106, the conduit 142 placing the needle lumen 140 in fluid communication with the fluid cavity 128. In some embodiments, the device 100 includes a sensor 144. In some embodiments, the sensor 144 comprises at least one of a pressure sensor, a force sensor, a touch sensors, a flow sensor, and/or an accelerometer. In some embodiments, the sensor 144 comprises a pressure sensor placed in fluid communication with the needle lumen 140 and/or the fluid cavity 128 and the pressure sensor provides measurements suitable for use as feedback during fluid injection. In some embodiments, the device 100 includes an actuator 146 that selectively causes the syringe 132 to inject fluid.

An example method of preparing a cornea tissue using a medical device in accordance with this detailed description may include stabilizing the cornea, inserting a needle or sharp fluid injection enabling object into a guide that leads to the cornea, and injecting fluid through the needle to separate layers of the cornea. Additional methods in accordance with this detailed description include methods of using a stabilization or guide device to prepare corneal tissue by making a bubble or hydrodissecting layers of the cornea. Methods contemplated in this detailed description also include methods of using a stabilization or guide device for separating layers of eye tissue by making a bubble or hydrodissecting layers of the eye, including but not limited to retina, lens nucleus, cataract, sclera, or other sectors of the globe.

An example method of preparing a cornea tissue using a medical device in accordance with this detailed description may include placing a cornea in the supporting structure, connecting the stabilizing body to the supporting structure to reduce or prevent movement of the eye, inserting a needle through the hollow channel feature, and injecting fluid through the needle into the cornea for separation of tissue layers. Methods contemplated in this detailed description also include methods of providing feedback on failure potential to eye bank technicians during tissue preparation, including but not limited to injection pressures and visual measurements.

In some embodiments, an ophthalmic medical device 100 for separating layers of eye tissue comprises a controlled fluid injection system. Controlled fluid injection systems that may be suitable in some applications are disclosed in the following United States patents all of which are hereby incorporated by reference herein: U.S. Pat. Nos. 9,113,843, 9,220,834, 9,241,641, 9,333,293, 9,352,105, and 9,457,140. The above references to U.S. patents in all sections of this application are herein incorporated by references in their entirety for all purposes. Components illustrated in such patents may be utilized with embodiments herein. Incorporation by reference is discussed, for example, in MPEP section 2163.07(B).

All of the features disclosed in this specification (including the references incorporated by reference, including any accompanying claims, abstract and drawings), and/or all of the steps of any method or process so disclosed, may be combined in any combination, except combinations where at least some of such features and/or steps are mutually exclusive.

Each feature disclosed in this specification (including references incorporated by reference, any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any incorporated by reference references, any accompanying claims, abstract and drawings), or to any novel one, or any novel combination, of the steps of any method or process so disclosed. The above references in all sections of this application are herein incorporated by references in their entirety for all purposes.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents, as well as the following illustrative aspects. The above described aspects embodiments of the invention are merely descriptive of its principles and are not to be considered limiting. Further modifications of the invention herein disclosed will occur to those skilled in the respective arts and all such modifications are deemed to be within the scope of the invention.

1. An ophthalmic medical device for separating layers of eye tissue comprising:
a) a supporting structure for supporting the eye; and
h) the supporting structure having at least one stabilizing feature that stabilizes the eye to reduce movement.

2.-10. (canceled)

11. The device in claim 1, wherein the supporting structure has a concave surface for receiving and/or holding a cornea and one or more visual cues for centering the cornea.

12. The device in claim 11, wherein the one or more visual cues for centering the cornea comprise a plurality of holes arranged in a pattern, the plurality of holes defining a first circle, the first circle being concentric with a second circle defined by an edge of the concave surface.

13.-38. (canceled)

39. An ophthalmic medical device for separating layers of eye tissue comprising:
a) a supporting structure for supporting at least a portion of an eye;
b) the supporting structure having at least one stabilizing feature that stabilizes the portion of the eye to reduce movement;
c) the supporting structure defining at least one hollow channel.

40. The device in claim 39, wherein the supporting structure has a concave surface for receiving and/or holding a cornea of the eye and one or more visual cues for centering the cornea.

41. The device in claim 40, wherein the one or more visual cues for centering the cornea comprise a plurality of holes arranged in a pattern, the plurality of holes defining a first circle, the first circle being concentric with a second circle defined by an edge of the concave surface.

42. The device in claim 41, wherein the hollow channel extends along a line that is parallel to a tangent line, the tangent line being tangent to the concave surface.

43. The device in claim 41, wherein the hollow channel extends at an angle less than or equal to 90 degrees relative to a reference plane, the reference plane being defined by the second circle.

44. The device in claim 43, wherein the reference plane has least a 3.6 degree tilt from a horizontal plane defined by a bottom surface of a base portion of the supporting structure.

45. The device in claim 39, wherein the supporting structure comprises a base portion and a stabilizing portion, and the stabilizing portion applies a mechanical force across a cornea contacting region, the cornea contacting region extending through a span that is equal to or less than 360 degrees around a periphery of a cornea when the cornea is received in a concavity defined by a concave surface.

46. The device in claim 39, wherein the supporting structure has a concave surface defining a concavity for receiving and/or holding a cornea of the eye and protrusions extending beyond the concave surface into the concavity, the protrusions being positioned for enabling a cornea to be supported proximate a needle entry point, the needle entry point being disposed proximate an end of the hollow channel.

47. The device in claim 39, wherein the hollow channel has a length, a diameter and an aspect ratio of length to diameter, the aspect ratio of length to diameter being greater than 3.

48. The device in claim 39, wherein the hollow channel has a diameter between 0.15 mm and 0.95 mm.

49. The device in claim 39, wherein the at least one stabilizing feature applies a sub-atmospheric pressure on a convex side of a cornea.

50. The device in claim 39, wherein the at least one stabilizing feature comprises a concave surface for receiving a cornea and at least one annular groove disposed about the concave surface, the annular groove extending 360 degrees along a circular path, wherein the annular groove is in selective communication with a source of sub-atmospheric pressure.

51. The device of claim 39, further comprising a needle fixing mechanism capable of selectively precluding movement of a needle relative to the hollow channel during injection of a fluid, the needle fixing mechanism comprising at least one of a set screw, a locking pin, a quick-release pin, a spring pin, a pin or a screw.

52. The device in claim 39, further comprising a fluid injector having a fluid cavity and an injectable fluid disposed in the fluid cavity, the injectable fluid being selected from the group consisting of water, saline, air, corneal storage media, tissue culture media, and balanced salt solution.

53. The device in claim 52, wherein the fluid injector comprises a syringe, the syringe comprises a syringe barrel and a syringe plunger, a distal portion of the syringe plunger being slidingly received in the syringe barrel, the syringe barrel and the syringe plunger cooperating to define the fluid cavity.

54. The device in claim 53, further comprising a needle having a distal end, a proximal end and a needle body extending between the distal end and the proximal end, the needle body defining a needle lumen, the needle lumen extending between the proximal end and the distal end of the needle, the needle lumen being in fluid communication with the fluid cavity.

55. The device in claim 39, wherein the hollow channel comprises a curved needle tract.
